(19) United States
(12) Patent Application Publication
(10) Pub. No.: US 2023/0250464 A1
(43) Pub. Date: Aug. 10, 2023

(54) BIOSENSOR APPARATUS AND METHOD OF USE THEREOF

(71) Applicant: THE ROYAL INSTITUTION FOR THE ADVANCEMENT OF LEARNING / MCGILL UNIVERSITY, Montreal (CA)

(72) Inventors: Juanjuan LIU, Montreal (CA); Sebastian Wachsmann Hogiu, Montreal (CA); Reza Abbasi, Montreal (CA); Meruyert Imanbekova, Montreal (CA)

(21) Appl. No.: 18/164,654

(22) Filed: Feb. 6, 2023

Related U.S. Application Data
(63) Continuation of application No. 63/307,492, filed on Feb. 7, 2022.

(51) Int. Cl. C12Q 1/26; B01L 3/00; G01N 21/76

(52) U.S. Cl. CPC C12Q 1/26; B01L 3/502715; G01N 21/76; B01L 2300/0654; B01L 2300/0645; B01L 2300/0819; B01L 2300/16; G01N 2333/90694

(57) ABSTRACT

There is described a biosensor apparatus. The biosensor apparatus generally has: a photodiode array having a first photon receiving face, a transparent substrate covering the first photon receiving face of the photodiode array, the transparent substrate having a second photon receiving face opposite the photodiode array, an electrically conductive coating covering the second photon receiving face of the transparent substrate, the electrically conductive coating being transparent and having two electrical contacts spaced apart from one another, and an analyte receiving area extending between the two electrical contacts of the electrically conductive coating.

### FIELD

The improvements generally relate to the field of biosensors, and more specifically to optical biosensors.

### BACKGROUND

Point-of-need testing has tremendous value in diagnostics (medical and non-medical) and more so in circumstances when a point-of-need device is low cost, portable and provides a ‘sample-in-answer-out’ solution. However, diagnostic technologies require quantitative readings which provide sufficient information to extra diagnostic level information. This necessitates specialized instrumentation which are unsatisfactory for miniaturization or are too costly for use in point-of-need applications. There always remains room for improvement.

### SUMMARY

Microfluidic technologies have been shown to provide certain advantages by integrating microchannels, chambers, pumps, and other components to manipulate small volume samples. These types of fluidic systems minimize consumption of reagents and provide a platform for permitting the integration of many analytical procedures into a single device. While microfluidic systems provide a way of compacting the fluidic pathways, the interrogation and measurement of the analyte of interest require for complex and costly external instrumentation, which can often become even more complex when retrofitted to a smaller fluidic apparatus.

Optical sensors are known to be versatile methods of interrogating and extracting key information from an analyte. There exists various ways of interacting with an analyte or a by-product which itself interacts with the analyte. This further depends on the application, the analyte itself, and the information desired. Some products release a definite amount of energy in the form of photons when they interact with analytes which permits the extraction of relevant information from the analyte. For instance, chemiluminescence (CL) reactions are complex multi-step reactions in aqueous conditions which emit light. Briefly, the reaction of a CL agent, such as luminol for instance, paired with an oxidant, like hydrogen peroxide (H₂O₂), generates an intermediate that exhibits CL emission. During this oxidation reaction, the luminol (e.g., 3-aminophthalhydrazide or 5-amino-2,3-dihydro-1,4-phthalazinedione) is oxidized into a dicarboxylate ion and loses molecular nitrogen while gaining oxygen atoms, producing 3-aminophthalate. The resulting 3-aminophthalate is in an excited state because the electrons in the oxygen atoms are elevated to higher orbitals. The electrons quickly return to a lower energy level, releasing a fraction of the excess energy as photons. This reaction occurs best with a catalyst, which may be an electric potential difference for instance, which oxidizes luminol and produces the excited state. The use of an electric potential difference as a catalyst for these types of reactions are known as electrochemiluminescence (ECL) and the oxidation luminol can be used to design biosensors exhibiting high sensitivity and a wide linear range of operation.

Providing the electrical potential (electromagnetic field) and capturing the optical information is no easy feat and more so in the context of a miniaturized fluidic system where a limited quantity of analyte and of CL agent is available. Recording and analyzing the ECL emission requires specialized photodetectors. Signal collection can be achieved using single-point photodetectors (such as photomultiplier tubes or avalanche photodiodes) due to their high gain, sensitivity, and linearity. However, these types of detectors cannot be used in portable or miniaturized devices due to their size, high-voltage supply, and power consumption, and cannot be easily integrated into multiplexed detection designs.

It was found that providing a biosensor having a photodiode array, such as a CMOS sensor for instance, with a substrate coated with a conductor forming an electrode which is capable of being integrated with a microfluidic system permits to overcome at least some of the issues in interrogating analytes and capturing optical signal related therefrom. The biosensor can provide a miniaturized, low cost, low-power system which can permit the interrogation of the analyte while providing an increased photon collection due to the close proximity of ECL reactions to the detector(s).

It is understood that the use of an electric potential difference for the purposes of oxidation of luminol, as described herein is provided as an example only and should not be construed as limitative in any way. Other uses of electrical potential (methods of providing an electromagnetic field) may be provided, with or without other analytes, without departing from the present disclosure.

It is understood that the term “interrogating” refers to any action, process, potential, interaction which can lead to the emission of an optical signal from a sample. The use of an electrical potential (e.g., electromagnetic field) for interrogation of a ECL solution is provided as an example only and should not be construed as limitative in any way.

In accordance with a first aspect of the present disclosure, there is provided a biosensor apparatus comprising: a photodiode array having a first photon receiving face, a transparent substrate covering the first photon receiving face of the photodiode array, the transparent substrate having a second photon receiving face opposite the photodiode array, an electrically conductive coating covering the second photon receiving face of the transparent substrate, the electrically conductive coating being transparent and having two electrical contacts spaced apart from one another, and an analyte receiving area extending between the two electrical contacts of the electrically conductive coating.

Further in accordance with the first aspect of the present disclosure, the biosensor apparatus can for example further comprise a body of material deposited onto the transparent substrate, the body having a recessed channel forming a fluidic channel when the recessed channel is received onto the analyte receiving area. In some embodiments, the body is made of polydimethylsiloxane (PDMS). In some embodiments, the fluidic channel has a height of approximately 143 μm, a width of approximately 650 μm, and a length of about 3.5 mm. In some embodiments, the fluidic channel is a microfluidic channel. In some embodiments, the fluidic channel can receive between about 1 and 10 μL of fluid, and most preferably between about 2 and 5 μL of fluid.

Still further in accordance with the first aspect of the present disclosure, the body can for example have an inlet and an outlet fluidly connected at opposite ends of the fluidic channel.

Still further in accordance with the first aspect of the present disclosure, the photodiode array, the transparent substrate and the electrically conductive coating can for example form a first detector assembly, the biosensor apparatus can for example comprise a second detector assembly identical to the first detector assembly, the first detector assembly and the second detector assembly both facing the analyte receiving area to receive photons therefrom.

Still further in accordance with the first aspect of the present disclosure, the first detector assembly and the second detector assembly can for example form a gap therebetween, the gap having a dimension ranging between 50 μm and 200 μm.

Still further in accordance with the first aspect of the present disclosure, wherein the photodiode array can for example be a complementary metal-oxide-semiconductor (CMOS) sensor. In some embodiments, the CMOS sensor can have an active area of at least 2 mm by 2 mm. In some embodiments, the CMOS sensor can have an active area of 3.67 mm by 2.73 mm.

Still further in accordance with the first aspect of the present disclosure, the electrically conductive coating can for example have a thickness between 3 nm and 500 nm, and most preferably between 10 nm and 300 nm.

Still further in accordance with the first aspect of the present disclosure, the electrically conductive coating can for example be an Indium Tin Oxide (ITO) coating.

Still further in accordance with the first aspect of the present disclosure, the transparent substrate can for example have a thickness between 50 nm and 200 μm, and most preferably between 100 μm and 150 μm. In some embodiments, the transparent substrate can have a thickness of approximately 127 μm.

Still further in accordance with the first aspect of the present disclosure, the transparent substrate can for example be a Poly Ethylene Terephthalate (PET) film.

Still further in accordance with the first aspect of the present disclosure, the PET film can for example form a 3-dimensional pattern having a plurality of protrusions protruding from the photodiode array. In some embodiments, the protrusions form a pyramidal shape, a triangular shape, a square shape, an hexagonal shape, a cuboid shape, a dome shape, or a combination thereof.

Still further in accordance with the first aspect of the present disclosure, the biosensor apparatus can for example further comprise an external voltage source applying an electrical voltage across the electrically conductive coating via the two electrical contacts.

Still further in accordance with the first aspect of the present disclosure, the biosensor apparatus can for example further comprise a pair of conductive connectors electrically connected between the two electrical contacts and the external voltage source.

Still further in accordance with the first aspect of the present disclosure, the photodiode array can for example be a 2-dimensional photodiode array.

Still further in accordance with the first aspect of the present disclosure, the transparent substrate can for example be an electrical insulator.

In accordance with a second aspect of the present disclosure, there is provided a method of optically detecting an analyte using a biosensor apparatus, the biosensor apparatus having a photodiode array, a transparent substrate atop the photodiode array, an electrically conductive coating atop the substrate, the electrically conductive coating being transparent and having two electrical contacts spaced apart from one another, and an analyte receiving area atop the electrically conductive coating between the two electrical contacts, the method comprising: the analyte receiving area receiving an analyte containing sample; generating an electromagnetic field across the analyte receiving area via the two electrical contacts, said electromagnetic field stimulating electrochemiluminescence (ECL) light emission in the analyte containing sample; and the photodiode array generating an electrical signal based on a detection of said ECL light emission.

Further in accordance with the second aspect of the present disclosure, the analyte containing sample can for example be one of liquid and gaseous.

Still further in accordance with the second aspect of the present disclosure, said generating the electromagnetic field can for example include applying an electrical voltage along the electrically conductive coating between the two electrical points.

Still further in accordance with the second aspect of the present disclosure, the electrical voltage can for example be of at least 5 V.

Still further in accordance with the second aspect of the present disclosure, said receiving the analyte containing sample can for example include flowing the analyte containing sample within a fluidic channel extending across the analyte receiving area.

In accordance with a third aspect of the present disclosure, there is provided a biosensor apparatus comprising: a photodiode array, a substrate atop the photodiode array, an electrically conductive coating atop the substrate, and an analyte receiving area atop the electrically conductive coating, the photodiode array having a field of view encompassing the analyte receiving area and extending across the substrate and the electrically conductive coating.

Still further in accordance with the third aspect of the present disclosure, when an electrical voltage is applied across the electrically conductive coating, analytes present at the analyte receiving area can for example emit electrochemiluminescence (ECL) light emission across the electrically conductive coating, across the substrate and towards the photodiode array.

In accordance with a fourth aspect of the present disclosure, there is provided a method of optically detecting an analyte, the method comprising: positioning an analyte in a gaseous or liquid solution, in an analyte receiving area; generating an electromagnetic field in the analyte receiving area, said electromagnetic field stimulating light emission by electrochemiluminescence (ECL); and generating an electrical signal based on the detection of said light emission.

In accordance with a fifth aspect of the present disclosure, there is provided a biosensor for multiplexed optical detection of biological and chemical analytes with high sensitivity and specificity, the biosensor comprising of: a substrate for sample holding; a microfluidic system for sample handling; and an optical detector.

In accordance with a sixth aspect of the present disclosure, there is provided a method of manufacturing the biosensor apparatus, the method comprising of: manufacturing a 3D pattern onto a 2D array of photodiodes, the 3D pattern having an electrically conductive coating deposited thereon; and implementing a microfluidic system atop the electrically conductive coating.

Many further features and combinations thereof concerning the present improvements will appear to those skilled in the art following a reading of the instant disclosure.

### DESCRIPTION OF THE FIGURES

In the figures,

FIG. 1A is a schematic cross-sectional view of an example of a biosensor apparatus, in accordance with one or more embodiments;

FIG. 1B is a schematic top view of the biosensor apparatus of FIG. 1A, in accordance with one or more embodiments;

FIG. 2 is a schematic side view of an example of the biosensor apparatus of FIG. 1A, showing optical capture of photons emitted by the biosensor apparatus, in accordance with one or more embodiments;

FIG. 3 shows example experimental results of the biosensor apparatus of FIG. 1A and a top bright field view of the biosensor apparatus, in accordance with one or more embodiments;

FIGS. 4A to 4E are oblique perspective views of components of the biosensor apparatus of FIGS. 1A and 1B at different steps of its assembly, in accordance with one or more embodiments;

FIG. 5A is a schematic side view of another example of a biosensor apparatus, in accordance with one or more embodiments;

FIG. 5B is a schematic oblique perspective view of the biosensor apparatus of FIG. 5A, in accordance with one or more embodiments;

FIG. 6 is a schematic side view of yet another example of a biosensor apparatus, with an external voltage extending along a fluidic channel, in accordance with one or more embodiments;

FIG. 7 is a schematic side view of yet another example of a biosensor apparatus, with an external voltage extending across a fluidic channel, in accordance with one or more embodiments;

FIG. 8 is a schematic side view of yet another example of the biosensor apparatus, with an external voltage extending across a fluidic channel, in accordance with one or more embodiments;

FIGS. 9A-9D show plots which characterize the biosensor apparatus of FIGS. 1A and 1B in the context of a first application, in accordance with one or more embodiments;

FIGS. 10A-10B show calibration graphs of the biosensor apparatus of FIGS 1A and 1B in the context of the first application, in accordance with one or more embodiments;

FIGS. 11A-11C show biosensing performance of the biosensor apparatus of FIG. 1A and 1B in the context of the first application, in accordance with one or more embodiments; and

FIGS. 12A1-12E show comparisons of the signal collection provided by biosensor apparatus in comparison to a commercial microscope and a smartphone, in accordance with one or more embodiments.

### DETAILED DESCRIPTION

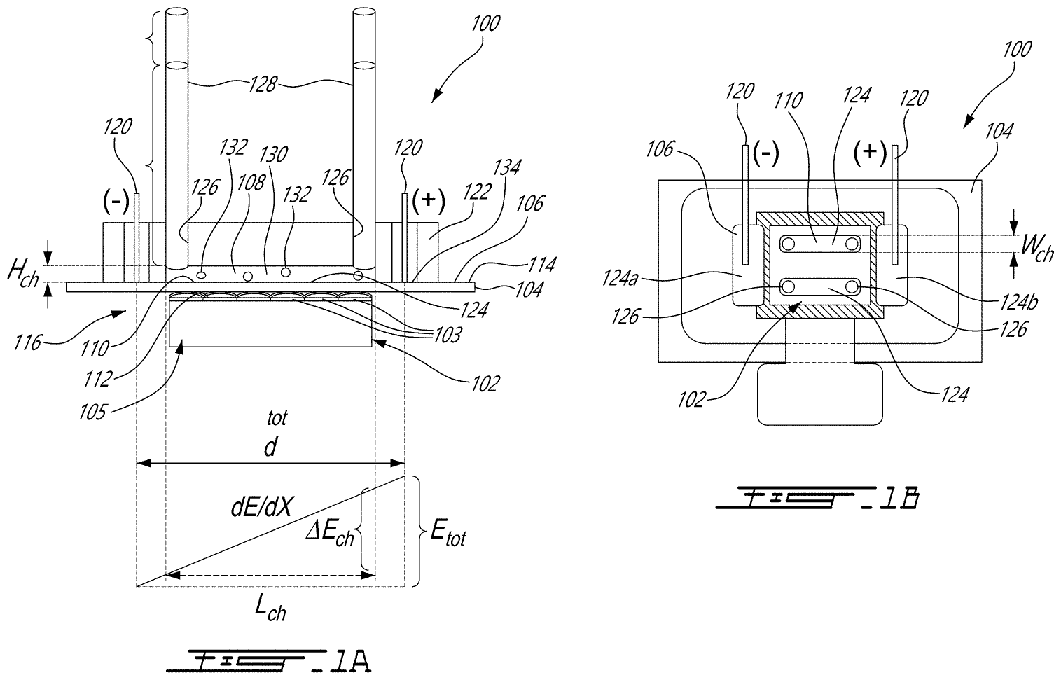

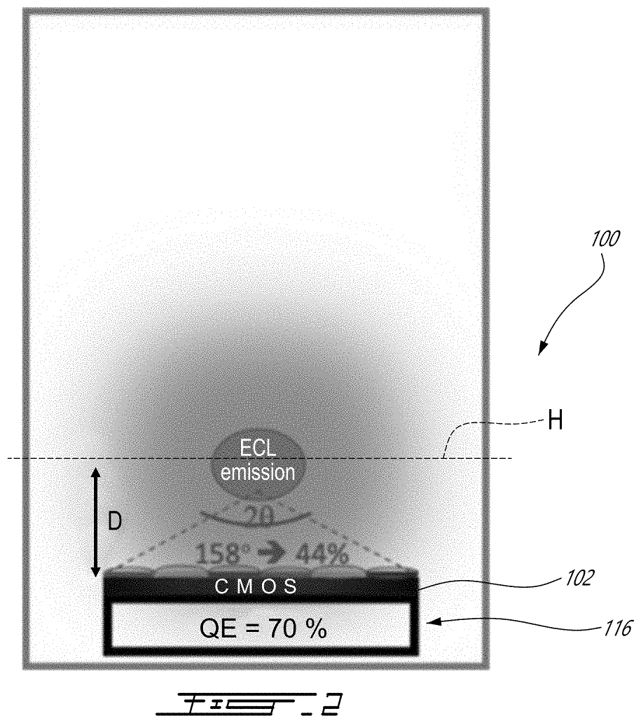

FIG. 1A shows a schematic side view of an example of a biosensor apparatus 100 while FIG. 1B shows a schematic top view of the example biosensor apparatus 100. For the purposes of clarity, the biosensor apparatus 100 will be described in the context of luminol-based electrochemiluminescence (ECL). It is understood that this exact application is an example only and should not be construed as limitative in any way.

The biosensor apparatus 100 in the embodiment shown in FIGS. 1A and 1B comprises a lens-less photodiode array 102. The photodiode array 102 includes a plurality of spaced apart photodiodes 103 which collectively form an optical detector 105. The photodiode array 102 is superposed to a transparent substrate 104 having the side opposite to the photodiode coated with a conductive material, which forms an electrically conductive coating 106. The electrically conductive coating 106 is transparent as well. A pair of microfluidic channels 108 are formed over the transparent substrate 104, which has an analyte receiving area 110. As will be understood, the photodiode array 102 has a first photon receiving face 112 which faces the microfluidic channels 108. Similarly, and for the purposes of clarity, the transparent substrate 104 further has a second photon receiving face 114 with faces the fluidic channels 108 (or the photon receiving face 112).

In this example, and as will be discussed below, the photodiode array 102 is a 5-megapixel backside-illuminated complementary metal-oxide-semiconductor (CMOS) sensor 116 from a commercially available Raspberry Pi™ module (OV5647, OmniVision technologies). The field of view (FOV) of the CMOS sensor 116 is 3.67 mm by 2.73 mm, which is the active area of the CMOS sensor 116, and the spatial resolution of the system is 1.4 μm, limited by the pixel size having a field of view (FOV) of 3.67 mm by 2.73 mm. However, it is understood that other CMOS sensors or chips from other suppliers and of any desirable size with different spatial resolutions or field of views may be used without departing from the present application.

The substrate 104 is a transparent Poly Ethylene Terephthalate (PET) film having been coated with Indium Tin Oxide (ITO). In other words, the electrically conductive coating of the transparent substrate 104 is made of ITO. As is perhaps best seen in FIG. 1A, the transparent substrate 104 is placed directly on the photodiode array 102 of the CMOS sensor 116 without the use of a lens. The side of the substrate 104 coated with the electrically conductive ITO is the second photon receiving face 114 of the substrate 104, which is opposite the side abutting with the CMOS sensor 116. The transparent material of the substrate 104, i.e., the PET, has a thickness between 50 μm and 200 μm, preferably between 100 μm and 150 μm, and more preferably of approximately 127 μm. The electrically conductive coating 106 of ITO has a thickness between 3 and 500 nm, preferably between 10 and 300 nm. In this embodiment, the transparent substrate 104 of PET forms an electrical insulation between the CMOS sensor 116 and the electrically conductive coating 106 of ITO. As will be discussed below, the electrically conductive coating 106 of ITO can be electrically connected with electrically conductive connectors 120. As such, the ITO coating 106 is electrically independent from the photodiode array 102.

In order to choose the optimal thickness of the ITO coating 106, the resistance and optical transparency of the coating 106 are to be considered. The difference in the coating resistance leads to a different current intensity that affects the gradient (see FIG. 1A, gradient under the schematic of the biosensor apparatus 110, which will be discussed below) of the electrical (electromagnetic) potential on the ITO coating 106, effectively forming an electrode. The different ITO coating thickness, on the other hand, results in variable electrical resistance of the ITO-coated film. More specifically, a thicker ITO coating leads to lower resistance. In the context of electrochemiluminescence (ECL) biosensors, ECL becomes more robust as the ITO resistance increases, reaching a peak at 100 ohm/square. On the other hand, since the ECL optical signal needs to be transmitted through the ITO-coated PET substrate 104, the transmittance is another critical factor in the overall performance of the biosensor apparatus 100. In this particular application, it was found that optimal transmittance of the chemiluminescence (CL) light (with wavelength around 419 nm in this application) was provided when the ITO coating had a resistance of 60 ohm/square. It was determined that this setup provided a transmittance almost 10% higher than the ITO coating with 100 ohm/square resistance. Given the relationship between the thickness of the ITO coating the resistance of the resulting layer, it can be more preferable to chose a thickness such as to provide a resistance of approximately 60 ohm/square.

As will be further discussed below, other materials may be contemplated without departing from the present application, and the ranges provided for ITO can be varied for other materials and coatings without departing from the present application. These ranges can be varied to provide a balance between the resistance of the coating, heat generation, possible failure of the material due to its burning, while not unduly inhibiting its optical transparency.

Still referring to FIG. 1A, the microfluidic channels 108 are provided on top of the coated substrate 104, or more specifically, on top of the ITO-coated PET substrate 104. The microfluidic channels 108 are formed between a body 122 and the ITO-coated PET substrate 104 and extend along a channel length L_{ch} with a channel width W_{ch} along an interrogation area 124 of the CMOS sensor 116, which in this case corresponds to the FOV of the CMOS sensor 116. As is perhaps best seen in FIG. 1B, for a single photodiode array which has a defined interrogation area 124, the body 122 can be formed to provide a plurality of fluidly distinct microfluidic channels 108. In this embodiment, the fluidic channels 108 are shown side by side, however, it is understood that other configurations may be provided without departing from the present application. A height H_{ch} of the microfluidic channel 108 is formed by the spacing between the ITO-coated PET substrate 104 and the upper most portion of the fluidic channel 108. In this particular example, the microfluidic channels 108 are identical, having a height of approximately 143 μm, a width of approximately 650 μm and a length of 3.5 mm, providing a volume of 3.3×10⁸ μm³ corresponding to a volume of the solution (electrolyte) of 3.3 μl in each of the fluidic channels 108. However, it is understood that these values may be changed based on the use and the application without departing from the present application. For instance, in an alternate embodiment, the microfluidic channels 108 may be provided to extend lengthwisely along a width of the interrogation area 124 of the photodiode array 102, providing a larger width and a shorter length for the microfluidic channels 108.

The body 122 is made of polydimethylsiloxane (PDMS) and sealed onto the ITO-coated PET substrate 104 via an oxygen plasma treatment. The PDMS body 122 further has openings 126 forming inlets and outlets at opposite extremities of each one of the microfluidic channels 108 permitting the reception of corresponding fluidic tubings 128 and the flowing of the sample 130 with the analyte 132 therein. The sealed microfluidic channels 108 form electrochemical cells whereas the conductive ITO-coated PET substrate 104 acts as a single electrode 134.

Still referring to FIGS. 1A and 1B, the electrically conductive connectors 120, which in this particular example are copper wires, are electrically connected to the electrically conductive coating 106 on opposite sides of the length of the microfluidic channels 108. More specifically, the conductive connectors 120 form an electrical contact 134 at a first lateral side 124a and at a second lateral side 124b of the interrogation area 124. A conductive silver epoxy was used at the electrical contact points to connect the conductive connectors 120 to the surface of the ITO-coated PET substrate 104 via cold soldering. It is understood that other engagement means may be used without departing from the present disclosure. These opposite conductive connectors 120 are electrically connected to a power supply, which creates a gradient of electric potential V along the fluidic channel 108, as is perhaps best seen in the lower part of FIG. 1A. The resistance of the ITO electrode creates a gradient of potential along the fluidic channel 108. When a specific voltage V is applied, the relative fraction of the potential difference inside the fluidic channel 108, forming the electrochemical cell, and the applied voltage corresponds to the relative length of the fluidic channel 108 and the distance between the conductive connectors 120. When the potential is large enough, a faradaic reaction co-occurs at both ends of the fluidic channels 108. In the case of fluidic channels 108 filled with carbonate electrolyte containing luminol, H₂O₂, and triton X-100, the oxidation of luminol occurs at the higher electric potential and the reduction of H₂O₂ occurs at the lower electric potential. Under these conditions, the luminol is oxidized at the higher electric potential of the fluidic channel 108 and generates an excited electronic state, which then relaxes to the ground state by emitting light with wavelengths centered at around 419 nm.

The voltage applied can vary depending on the resistance of the ITO coating. In this particular example, with a coating providing a generally uniform resistance of approximately 60 ohm/square, it was determined that voltages varying from 2.5 to 5 V may be applied. A voltage of 2.5 V was sufficient to drive the desired ECL reaction, and thus the faradaic reaction occurs simultaneously at both ends of the fluidic channels 108. Since this reaction is driven by electric potential difference, increasing the voltage results in increasing the emission until 5 V. The blue ECL emission of luminol was observed at the positive side of the fluidic channel 108 starting at around 2.5 V. By increasing the voltage difference, the emission region of the channel gradually expands toward the center of the fluidic channel, allowing the ECL reaction to occur over a greater surface area. It is understood that different voltage values may be used with different concentration ranges for the fluidic solution without departing from the present application. The voltages used may be higher than 5 V in certain applications, such as to shift the concentration range to the lower limits of detection. However, higher voltages can result in higher electrical currents and material temperature, which alter the enzyme activity and can damage the ITO-coated PET substrate 104.

As is perhaps best seen in FIG. 2, showing a schematic of the ECL emission within the biosensor apparatus 100, where the body, electrically conductive coating and substrate are hidden, the proximity of the reaction to the photodiode array 102 leads to a large solid angle for photon collection one it is emitted by the ECL reaction. This detection solid angle is larger than the solid angle offered by most microscope objectives, which are limited by the working distance and aperture of the objective lens. Considering the whole solid angle as 360 degrees, and for the purposes of the example assuming that the measuring distance is approximately D=300 μm and the length of the CMOS sensor 116 is 3200 μm as discussed above, the current configuration can collect between 40% to 50% of the photons emitted by the CL reaction, preferably approximately 44% of the photons emitted by the CL reaction.

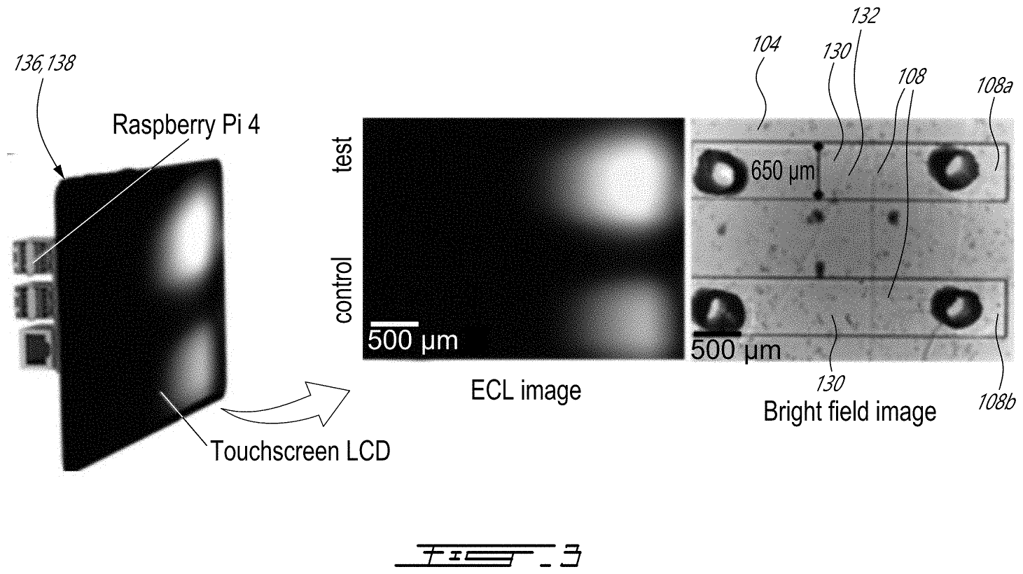

Referring to FIG. 3, when the fluidic channels 108 are filled with the ECL solution 130 and a voltage is applied, the photons emitted by the CL agent pass through the ITO-coated PET substrate 104 and are captured by the photodiode array, which permits to measure and quantify the reaction occurring in the fluidic channels 108. In this embodiment, and as described above, a pair of fluidic channels 108 are provided over the photodiode array, of which a bright field image is shown on the right-hand side of FIG. 3. Both the fluidic channels 108 are filled simultaneously with the ECL solution 130, while one of the fluidic channels 108 further contains the analyte 132 therein, forming a test channel 108a. The fluidic channel 108 which does not contain the analyte 132 plays the role of a control channel 108b, where the ECL emission in the control channel provides the baseline and the ECL intensities are obtained by subtracting the intensity recorded in the control channel 108b from the one recorded in the test channel 108a. As can be seen in FIG. 3, the signal captured in the test channel 108a is noticeably larger than that captured in the control channel 108b. It is understood that in alternate embodiments, a single control channel may be used as a baseline for more than one test channel comprising the same or different analytes without departing from the present application. In yet another embodiment, the test channel 108b may be omitted and the ECL signal difference between two different channels having different amounts of the analytes may be compared. For instance, in an alternate embodiment, it may be desirable to determine if a certain amount of an analyte is found in excess to a normal range, in which case the control channel may include an amount of analyte which is deemed within a normal range, while the test channel(s) may be sampled with an unknown amount of the analyte. The signal captured by the photodiode array 108 may be received and processed by a computer 136 such as to provide a value representative of the presence of an analyte or of a quantity of a certain analyte. The computer 136 can comprise a processor and a non-transitory memory having instructions stored thereon which when executed by the processor performs some preprogrammed steps. This may be done using a portable headless computer 138 such as a Raspberry Pi™ 4, for instance.

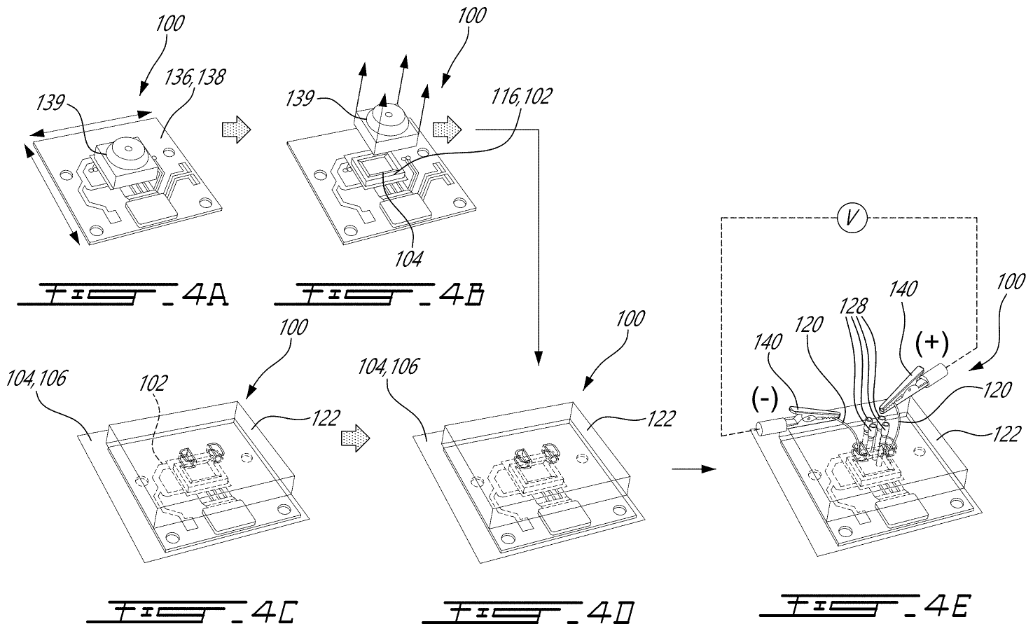

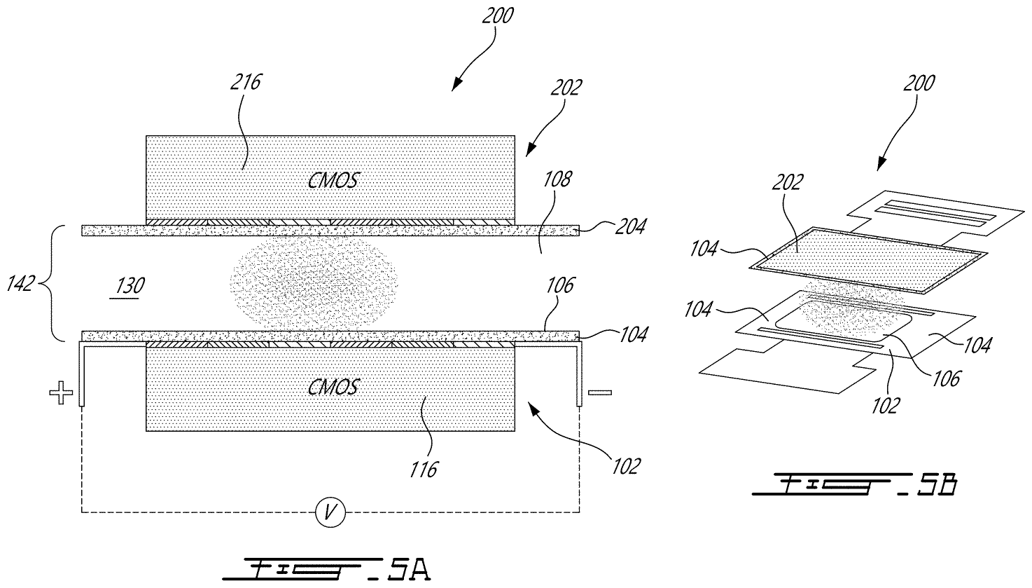

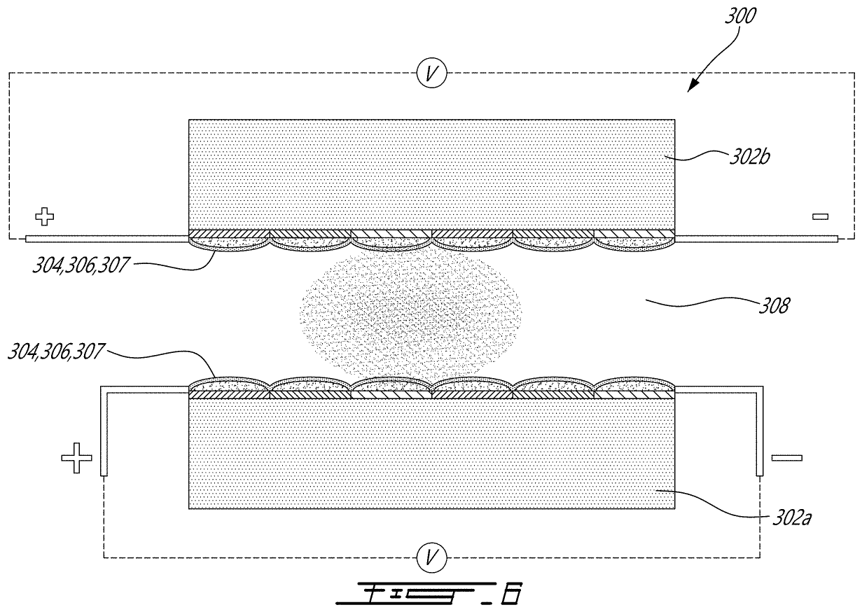

Attention is now brought to FIGS. 4A to 4E showing the components of the biosensor apparatus 100 of FIGS. 1A and 1B at different steps of its assembly. As is best seen in FIG. 4A, in this example the photodiode array 102 is a camera module of a portable headless computer 138 such as a commercial Raspberry Pi™, which by default comprises a lens 139 aligned with the CMOS sensor 116. This lens 139 and the infrared filter (not shown) are removed to expose the CMOS sensor 116 of the optical module as shown in FIG. 4B. In parallel, the biosensor apparatus 100 is fabricated by coating the PET substrate 104 with an electrically conductive coating 106 of ITO, and engaging the body 122 of a microfluidic system onto the ITO-coated PET substrate 104, as shown in FIG. 4C. The CMOS sensor 116 is then engaged on the optical detector in such a way that the microfluidic channels are aligned with and extend within the field of view of the CMOS sensor 116, forming an interrogation area which overlaps with the area exposed to the CMOS sensor 116, as shown in FIG. 4D. Lastly, the fluidic tubings 128 are engaged with the openings 126 of the microfluidic body 122 to permit the insertion and pumping of the solution 130 with or without the analyte 132, and the conductive connectors 120 are engaged with electrically conductive coating 106 to provide the electrical potential gradient (forming an electromagnetic field). The conductive connectors 120 may be electrically connected to an external voltage V via any suitable means, such as alligator clips 140, for instance, as shown in FIG. 4E.

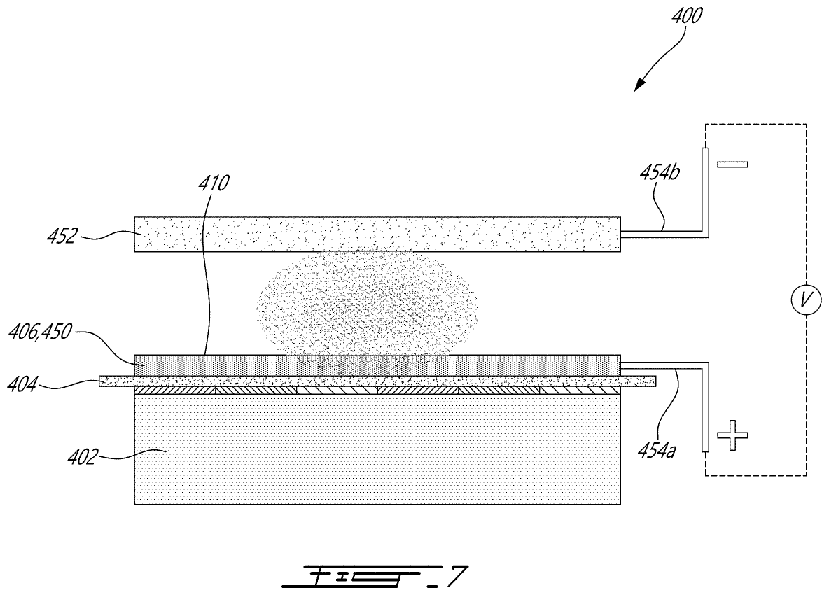

Attention is now brought to FIGS. 5A and 5B. As the ECL reaction generates the emission of light in all directions, it can be desirable to provide a biosensor apparatus 200 which comprises the photodiode array 102 (hereinafter referred to as “the first photodiode array 102”) and also an additional photodiode array 202 (hereinafter referred to as “the second photodiode array 202”) such as to increase the amount of the optical emission provided by the reaction. As depicted, the first photodiode array 102 is part of a first detector assembly whereas the second photodiode array 202 is part of a second detector assembly. Both the first and second detector assemblies face the analyte receiving area, and more specifically the fluidic channel 108 to receive photons therefrom. In this embodiment, the first detector assembly comprises the first photodiode array 102, the PET substrate 104, and the conductive ITO coating 106 deposited onto the PET substrate 104, as was disclosed in relation to FIGS. 1A and 1B. However, the second detector assembly has the second photodiode array 202 which faces the first photodiode array 102 found in the first detector assembly. The first and second detector assemblies are spaced apart from one another and form a gap 142 within which the fluidic channel 108 extends. In this manner, the CL solution 130 with or without the analyte, is provided in the gap 142 extending between the first and second detector assemblies and the emission provided by the ECL reaction may be captured by both the first and second photodiode arrays 102 and 202. With reference to FIG. 2, the photons which are generally emitted in a direction below the horizontal reference line H would mainly be captured by the first photodiode array 102 in the first detector assembly of the biosensor apparatus 200, while the photons which are generally emitted in a direction above the horizontal reference line H would mainly be captured by the second photodiode array 202 in the second detector assembly of the biosensor apparatus 200. The gap 142 between the two opposed photodiode arrays 102 and 202 defines the fluidic channel 108. In some embodiments, the gap 142 is between 50 μm and 200 μm.

The second photodiode array 202 of the second detector assembly of the biosensor apparatus 200 is covered by a substrate 204 which, in this example, is the same PET substrate 104 provided in the first detector assembly. However, the substrate 204 is not provided with an electrically conductive coating and is not connected to an external voltage V, as is the case with the electrically conductive coating 106. It is understood that in alternate embodiments, the electrically conductive coating 106 may be provided in the substrate found in the second detector assembly instead of the first detector assembly of the biosensor apparatus 200 or on both the first and second detector assemblies without departing from the present application.

Still referring to FIGS. 5A and 5B, the microfluidic system (not shown) differs in that the body does not cover the fluidic channels, but instead forms the lateral limits of the fluidic channel. The PDMS is formed with various window like openings, which delimits the fluidic channels laterally, between an inlet and an outlet, while the substrates, with or without the ITO coating, further delimit the first and second detector assemblies of the fluidic channel 108.

In this embodiment, the photodiode arrays 102 and 202 are identical CMOS sensors 116 and 216 and the substrate are the same PET material. It is understood, however, that in alternate embodiments, the CMOS sensors 116 and 216 may be different sizes, manufacturers and have different spatial resolutions without departing from the present application. Similarly the uncoated substrate 204 may differ in material, thickness, size and transmittance with respect to the substrate 104, as suitable for the application without departing from the present application.

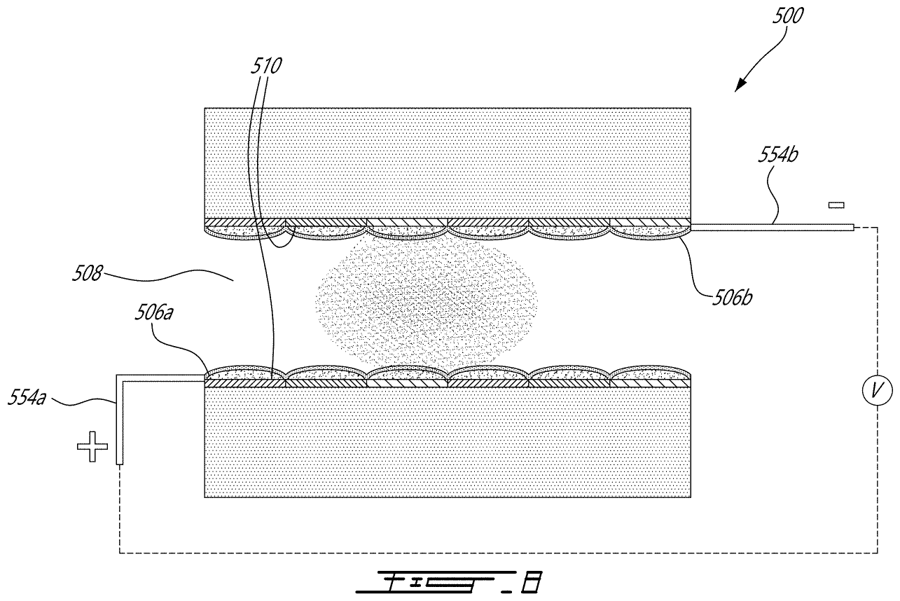

Attention is brought to FIG. 6, showing another example of the biosensor apparatus 300 having a first detector assembly with a first photodiode array 302a and a second detector assembly with a second photodiode array 302b, forming a fluidic channel 308 therebetween. In this embodiment, the PET substrates 304 are provided with protrusions 307 forming a 3-dimensional pattern directly over the respective first and second photodiode arrays 302a and 302b. The 3-dimensional pattern shown in FIG. 6 is a dome-like shape. The protrusions 307 are further provided with electrically conductive coatings 306 of ITO thereon, which follow the respective 3-dimensional pattern of the protrusions 307. It is understood that the 3-dimensional substrate patterns may be altered to any suitable shape without departing from the present application. For instance, in an alternate embodiment, the 3-dimensional pattern is a cuboid shape. In yet another alternate embodiment, the 3-dimensional pattern is a pyramidal shape which has its base in contact with the photodiode array forming a square area, rectangular area or a hexagonal area, to name some examples. Other shapes may be used without departing from the present application.

Still referring to FIG. 6, in this embodiment, the first and second photodiode arrays 302a and 302b are both coated with the electrically conductive ITO material and electrically connected to a respective or same external voltage V. The polarity of the external voltage V applied along the length of the fluidic channels 308 corresponds to each other for the both the first and second detector assemblies, such as to form corresponding gradient of potential along the length of the fluidic channels 308. Provided the increase capacity of capturing the optical emission of the ECL reaction, and the increase gradient of potential provided by the combined voltage of the upper and lower electrically conductive ITO coatings 306, the voltage applied to each one of the detector assemblies of the biosensor apparatus 300 in the embodiment of FIG. 6 can be lower than that of the configuration of FIG. 1A, for instance, while providing satisfactory results.

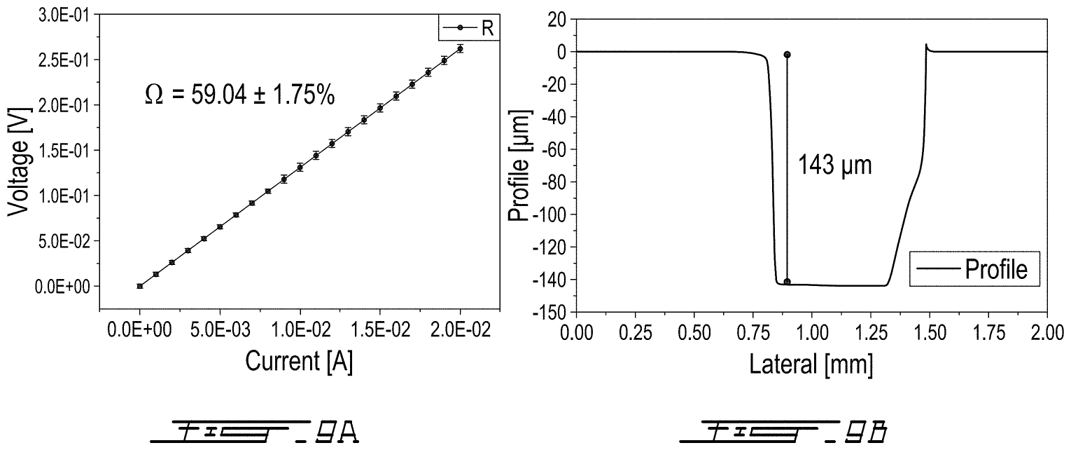

Attention is now brought to FIG. 7 showing a schematic side view of yet another example of a biosensor apparatus 400 which comprises two electrodes 450 and 452. In this embodiment, the electrically conductive coating 406 is connected to a first terminal 454a of an external voltage V via one of the electrodes 450. The other electrode 452 is provided in the form of a conductive layer provided across the fluidic channel 408, offset and separate from the conductive coating 406, is connected to the opposite terminal 454b of the external voltage V. In this form, the external voltage V provides an electrical potential across the analyte receiving area 410 within the fluidic channel 408.

It is understood that in alternate embodiments, a second photodiode array may be placed on the opposite side of the fluidic channel 408, as was discussed in FIGS. 5A and 5B or FIG. 6, and that the conductive layer may be provided with a second electrically conductive coating on a substrate placed on the second photodiode array.

Attention is brought to FIG. 8, which shows a schematic side view of yet another example of a biosensor apparatus 500 which comprises two electrodes. In this embodiment, a first electrically conductive coating 506a of the first detector assembly is connected to a first terminal 554a of an external voltage V, while a secondary electrically conductive coating 506b, extending on the second detector assembly, is connected to the second terminal 554b of the 25 external voltage V. In this form, the external voltage V provides an electrical potential across the analyte receiving area 510 within the fluidic channel 508.

### EXAMPLES AND PERFORMANCE ANALYSIS

For the purposes of completeness, an experimental methods and results for example analytes will be discussed.

The performance of the single electrode system discussed with reference to FIGS. 1A and 1B is being made by detecting uric acid (UA) in artificial body fluids, including urine and saliva. UA is an end product of metabolic breakdown of purine nucleotides and has long been used as a biomarker for assessing physiological health. Gout, hyperuricemia, Lesch-Nyhan syndrome, cardiovascular disease, kidney illness, and other disorders are all linked to abnormal UA levels. By combining a microfluidic platform on a CMOS chip, in this case a CMOS sensor, as discussed herein, we are able to demonstrate sensitive detection of UA via single electrode ECL.

The electrolyte used for ECL measurements was a mixture of 0.1 M sodium bicarbonate and 1 mM luminol. Specifically, to prepare the electrolyte, 0.1 M sodium bicarbonate was prepared, and the pH was adjusted accordingly by adding 1 M NaOH. It was then mixed with 10% v/v 10 mM luminol that was prepared in NaOH (0.1 M) to obtain a final concentration of 1 mM for luminol. Further, the analytes (H₂O₂ or UA) were added to this electrolyte for detection. The pH used for measurements was 10.7 unless otherwise stated.

For H₂O₂ detection, 10 mM H₂O₂ was prepared by diluting the concentrated H₂O₂ solution (9.8 M) in water and then mixed with the prepared electrolyte containing carbonate buffer and luminol. To create the calibration curve for H₂O₂ detection, different concentrations of H₂O₂ were prepared in the electrolyte.

Similarly, for UA detection, 1 mM UA was prepared in the electrolyte containing carbonate buffer and luminol for further dilutions. Further, different concentrations of UA from 25 μM to 1000 μM (25, 50, 100, 200, 300, 500, 800, and 1000 μM) were prepared by diluting the stock solution of 1 mM UA in the same electrolyte with carbonate buffer and luminol. The uricase solution of 1 mg/ml was prepared using NaOH (0.001 M, pH 11) and kept at −20° C. for further use. To evaluate the selectivity for UA detection, interfering molecules, i.e., ascorbic acid (5 μM), creatinine (10 μM), and glucose (50 μM) were added to the prepared UA solution for ECL measurement. 1% v/v of triton X-100 was added to the final solutions before each experiment to improve mixing of components.

To prepare the solutions for simulated sample analysis, artificial body fluids (saliva or Surine™) were mixed with the electrolyte containing carbonate buffer (0.1 M) and luminol (1 mM) at two different ratios (1:9 and 1:1). Further, 50 μM or 300 μM of UA was prepared in the mixture of the electrolyte and artificial body fluids using 1 mM and 6 mM stock solutions of UA, respectively, and the ECL intensities were captured. By using the calibration equation of UA detection, the concentration of UA corresponding to the obtained intensity (average of 3 experiments) was calculated, and the recovery rate was reported by comparing the measured concentration with the known (added) concentration.

In this specific example, the PDMS body of the microfluidic device was built by soft lithography. Briefly, the SU-8 photoresist on silicon wafer was exposed to 365-nm UV light using a mask aligner (the OAI Model 200) to create the positive master mold. Then, PDMS prepared in a 10:1 (monomer: crosslinker) ratio was used to replicate the pattern. This device was then sealed with the ITO-coated PET using oxygen plasma treatment (Diener Electronic, PICO) at 60 W for 2 minutes, followed by 30 minutes of incubation at 70° C. for irreversible bonding.

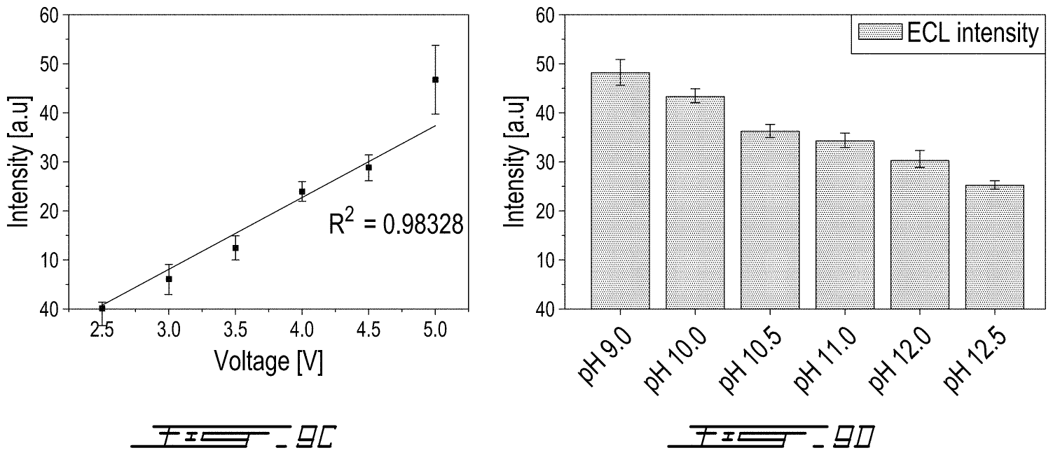

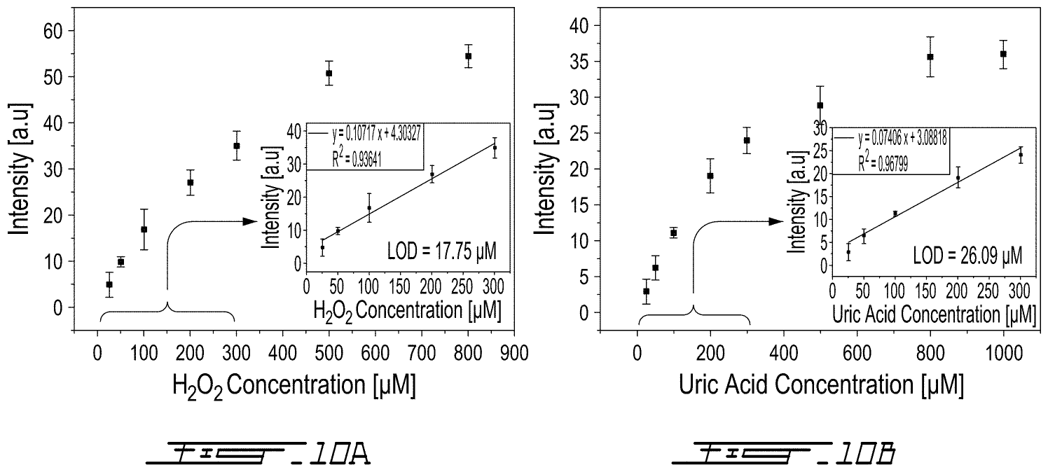

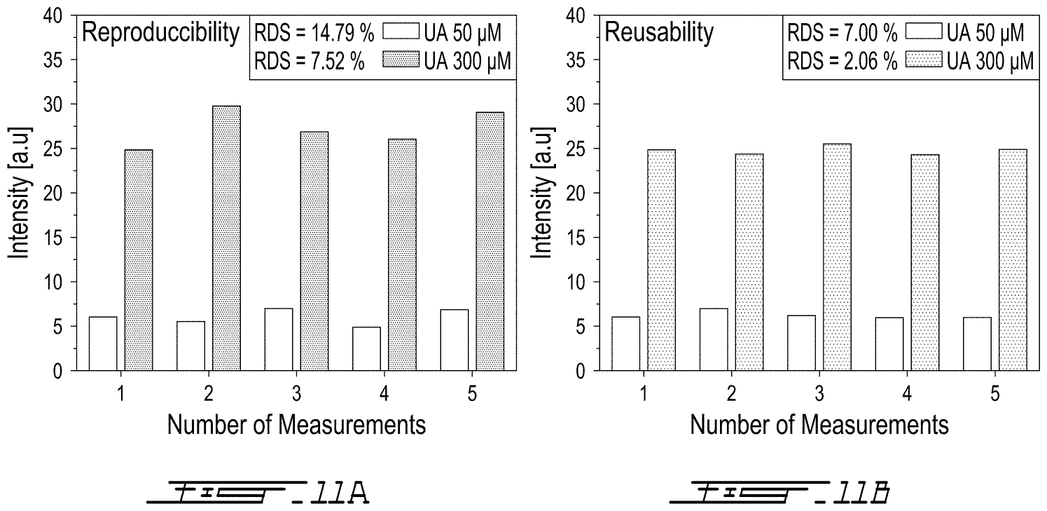

The ITO-coated PET substrate comprises of two distinct layers: a PET substrate with a thickness of 127 μm and an ITO coating with a thickness ranging from 10 to 300 nm. The ITO coating was selected to provide a resistance of 60 ohm/square. The resistivity of the ITO coated PET is shown in FIG. 9A. The sheet resistance was calculated using the following equation when the distance used for voltage measurement is at least twice the thickness of the film:

$${R}_{s} = k \frac{Δ V}{I} ,$$

where Rs is the sheet resistance, k is the geometrical correction factor which is 4.532 for the aforementioned experimental setup, ΔV is the measured voltage, and I is the applied current. As a result (FIG. 9A), the sheet resistance of the ITO-coated PET is 59.04 ohm/square with a relative standard deviation (RSD) of 1.75%, which indicates good uniformity. The biosensor apparatus was then connected to a digital power supply (e.g., Sky Toppower STP6005) using alligator clips and copper wires.

For UA detection, a final concentration of 200 μg/ml of uricase was added to the sample solution and incubated for 10 minutes before applying voltage. For the ECL experiments, the sample was transferred to the channels through the inlets using a syringe and a 30 G needle. The sample filled the fluidic channels as well as both inlets and outlets.

The measurements were performed in complete darkroom. A voltage of 5 V was applied to the device and the ECL emission was observed and recorded using a Raspberry Pi™ 4 computer and then analyzed via Fiji ImageJ to extract grey values corresponding to the detected photons. The measured grey values of the control channel were subtracted from those recorded for the test channel and the results were presented. Other computer or computing devices can be used in other embodiments.

In order to study the reusability (repeatability, using the same device), and reproducibility (using different devices), and selectivity (in the presence of interfering molecules), 50 and 300 μM of UA were used for ECL measurements.

For controlling the imaging parameters, a Python script was written, and the following conditions were applied: resolution was set to 2592×1944 pixels (full field of view), framerate was set to ⅙ s, shutter speed was set to a 6 s exposure time, ISO was set to 800 (gain 8×). Thirty seconds of sleep time was applied before each experiment to give the camera an appropriate time to adjust the parameters.

During the experiment, the channels are filled with the reagents at maximum capacity, i.e., 3.3 μl of mixed luminol, H₂O₂, and carbonate buffer. The 3.3 μl of electrolyte inside each fluidic channel contributes to the ECL emission. This amount of electrolyte corresponds to 3.3 nmol of luminol that emits light in the oxidation reaction. The height profile of the microfluidic channel is shown in FIG. 9B.

H₂O₂ was used to measure the ECL intensity at different voltages ranging from 2.5 to 5 V. As shown in FIG. 9C, a linear relationship between the intensity and the voltage was obtained as expected from the following equation:

$$Δ {E}_{ch} = {E}_{tot} ( \frac{{l}_{ch}}{d} ) ,$$

where ΔE_{ch} is the potential difference inside the electrochemical cell, Eₜₒₜ is the voltage applied by a power supply, I_{ch} is the length of the channel and d is the distance between wires, as illustrated in FIG. 1A. When ΔE_{ch} is large enough, the faradaic reaction co-occurs at both ends of the channels, with oxidation of luminol at higher electric potential and reduction of H₂O₂ at lower electric potential. Under these conditions, the luminol is oxidized at the higher electric potential of the channel and generates an excited electronic state, which then relaxes to the ground state by emitting light with wavelengths centered at around 419 nm (blue light). When Eₜₒₜ is more than 2.5 V, the ΔE_{ch} is sufficient to drive the reaction, and thus the faradaic reaction occurs simultaneously at both ends of the fluidic channels. Since this reaction is driven by electric potential difference, increasing the voltage results in increasing the emission until 5 V. The blue ECL emission of luminol was observed at the positive side of the channel starting at around 2.5 V. Increasing the value of Eₜₒₜ, the emission region of the channel gradually expands toward the center, allowing the ECL reaction to occur over a greater surface area. It is worth mentioning that above 5 V, the luminescence signal became saturated and the CMOS sensor was unable to process the actual data as it was above its full-well and maximum charge transfer capacity. Saturation of the CMOS detector limits the linear range and the performance of the device in the saturation range is not reliable. Thus, ECL experiments above 5 V have not been analyzed. Eventually, the device will operate at its optimum voltage of 5 V, which is a safe voltage for the operator and produces the largest signal for the concentration range selected for this application. While it is technically possible to shift the concentration range to the lower limits of detection by increasing the voltage, higher voltages result in higher electrical currents and material temperature, which alter the enzyme activity and can damage the ITO-coated PET substrate. In addition, at higher potentials, the oxidation of water takes over as the major process, and a significant amount of gas evolution is frequently observed due to the generation of molecular O₂.

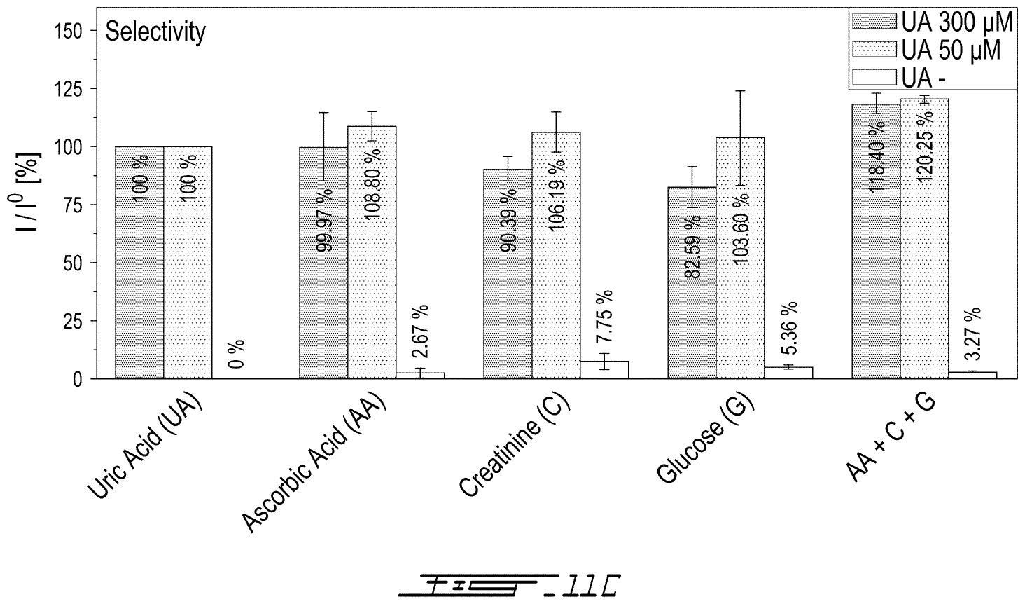

ECL intensities were recorded with H₂O₂ in electrolytes at different pH values. The results revealed that raising the pH decreases the intensity of the ECL, shown in FIG. 9D. In these experiments pH 10.7 is chosen.The limit of detection from these experiments for H2O2 and UA are shown FIG. 10. The limit of detection (LOD) was calculated according to LOD set to 3 SD/S, where SD is the standard deviation of the blank measurements when there is no analyte, and S is the slope of the linear equation obtained from the calibration plot.

The ECL intensity corresponding to H₂O₂ at different concentrations was reported. At least three experiments were conducted for each concentration, and based on the average intensity of each concentration, the relationship between the H₂O₂ concentration and the ECL intensity, a calibration plot was presented (FIG. 10A). A linear detection range from 25 to 300 μM was obtained with a coefficient of determination R² of 0.986, and LOD for H₂O₂ was calculated to be 17.75 μM.

To explore the capability of the biosensor apparatus to be employed in the detection and screening of an analyte that is a biomarker of several diseases, UA was investigated. UA is a product resulting from purine metabolism in the human body. Elevated UA levels in urine or serum can impair renal function and may be used as indicator of gout, cardiovascular and renal disease, hypertension, and other conditions. Low UA levels have been linked to molybdenum insufficiency, copper toxicity, and the progression of multiple sclerosis. As a result, the detection of UA in human physiological fluids is critical for diagnosing individuals with diseases linked with abnormal purine production and catabolism.

The oxidation of UA in the presence of the enzyme uricase that produces H₂O₂ is described in the following equation:

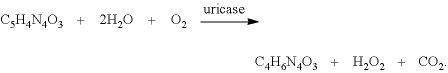

H₂O₂ is the product of the enzymatic oxidation reaction of UA, as such the luminol-H₂O₂ system is being used to detect UA.

Following the same procedure detailed for H₂O₂ detection, the calibration plot of UA was obtained by recording ECL intensities of UA at different concentrations (See FIG. 10B). The results show a linear detection range for UA from 25 μM to 300 μM with a coefficient of determination R² of 0.968, and a LOD of 26.09 μM.

To further explore the efficiency of the reaction of the equation above, the calibration plots (FIGS. 10A and 10B) for H₂O₂ and UA were compared to each other. Theoretically, this reaction should produce as much H₂O₂ as the stoichiometric ratio of H₂O₂ to UA (limiting reagent) indicates. The molar ratio between UA and H₂O₂ in the balanced equation is 1 to 1. As a result, in a constant volume, by oxidation of a certain concentration of UA in the reaction, the same concentration of H₂O₂ should be released. However, the amount of H₂O₂ actually produced by the reaction is usually less than the theoretical yield and is referred to as the actual yield. Thus, the efficiency of the reaction in equation 3 is defined as:

Efficiency of the reaction (percent yield)=(actual yield/theoretical yield)×100.

As shown in FIG. 10B, any given amount of UA added to the reaction solution correlates with an intensity value proportional to the actual amount of H₂O₂ produced, which itself can be calculated from FIG. 10A. On the other hand, based on the stoichiometric ratio of chemical reaction equation, the theoretical amount (mol) of produced H₂O₂ is equal to the amount of UA (mol) added to the solution. Hence, the efficiency of every experiment (i.e., every intensity value) can be calculated by dividing the actual concentration of produced H₂O₂ by the concentration of uric acid added. In other words, the actual yield and theoretical yield can be calculated from FIG. 10B and FIG. 10A, respectively. By comparing the results from calibration curve of UA and H₂O₂ (FIG. 10A and FIG. 10B), the efficiency of the reaction for each given concentration was calculated and then the average of all efficiencies in the linear concentration range was obtained to be 57.1%. In addition to some experimental errors, there are often losses as a result of an incomplete reaction, and unwanted side reactions such as the conversion of the H₂O₂ to the water due to the instability of the H₂O₂ at low concentrations and at room temperature.

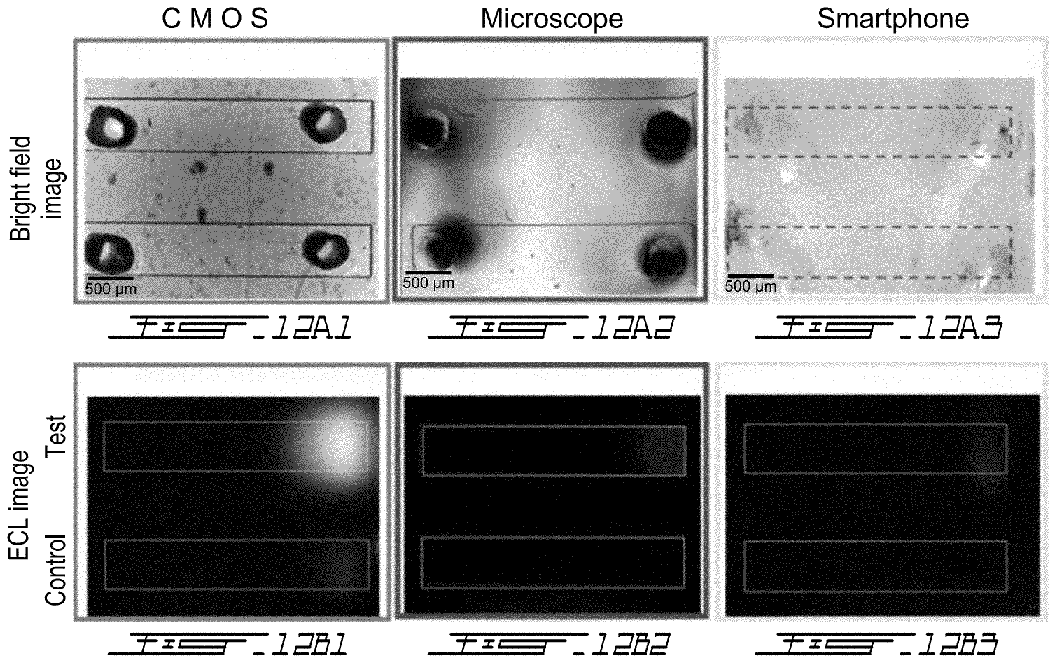

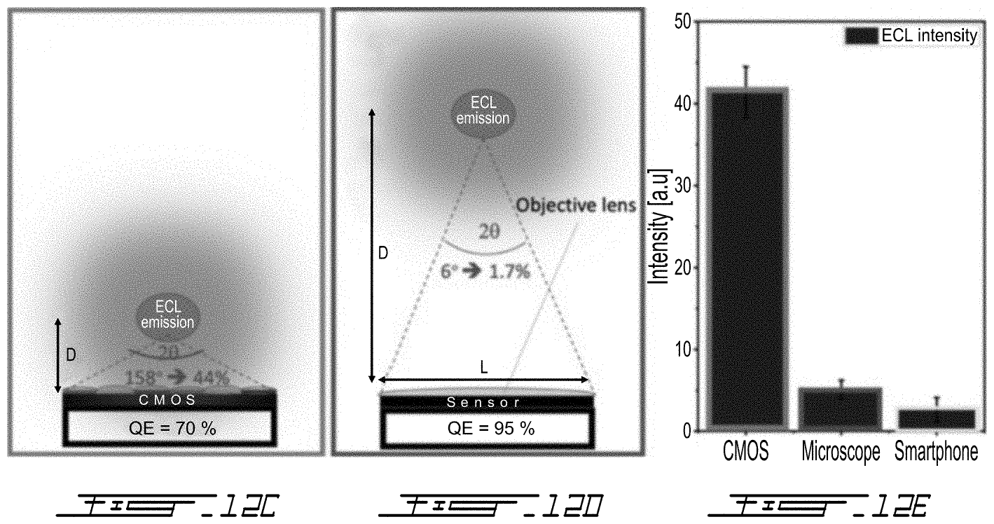

The reproducibility of different devices and the reusability of one device in multiple experiments are characteristics of a biosensor apparatus permits estimating its potential for practical applications. Hence, these parameters were studied, and the results are shown in FIGS. 11A1 to 11E.

Specifically, to evaluate the reproducibility of the fabrication process, five different devices were examined to detect 50 μM and 300 μM of UA each, as the low and the high concentration within the reported linear range of this device, in the electrolyte solution. The ECL intensities are collected, and the RSD of the intensity for these five experiments was calculated to be 14.79% for 50 μM of UA and 7.52% for 300 μM of UA. These numbers indicate good reproducibility for the detection of UA at different concentrations in the linear detection range. The observed variation is due to several factors. On one hand, the resistivity of the ITO PET substrate differs slightly between devices with a tolerance of approximately 1.75% (FIG. 9A). In addition to the ITO resistivity, the distance between the two conductive connectors (FIG. 1A) can also introduce some errors. Another source of error that can contribute to the variability of both reproducibility and reusability is the efficiency of the oxidation reaction of UA.

The manufactured device could technically be cleaned and reused. In order to test this practicality of this approach, the reusability (repeatability) of the same device for multiple experiments was then studied by measuring the SE-ECL intensities of 50 and 300 μM of UA. The ECL signal was recorded using the same device with fresh electrolyte solution each time for 5 times. The relative standard deviation (RSD) was calculated to be 7.00% and 2.06% for 50 and 300 μM of UA, respectively. After 5 experiments, the ECL intensity has gradually decreased, and after the 9th experiment, a dramatic fall in the ECL intensity was observed (results not shown). This is probably due to the damage of the ITO coating as a result of the passing current and induced heat, which consequently leads to decreased conductivity. However, since the device is inexpensive (the cost of the CMOS sensor is 1$ and the approximate cost of the microfluidic device is 3$), it can be used as a disposable device, and its reusability might not be an issue of significant concern.

There are various chemicals in body fluids that might interfere with the ECL detection of UA. For example, glucose, ascorbic acid (AA), and creatinine, which are concurrently present in body fluids, may interfere with the detection of UA. Therefore, the selectivity of the fabricated device in the presence of glucose, AA, and creatinine was studied by detecting 50 and 300 μM of UA in the electrolyte solution with and without interference. Concentrations of these interfering molecules were chosen based on their typical physiological concentrations in human saliva.

ECL UA intensities were recorded in the electrolyte with (I on the y-axis in FIG. 11C) and without interfering molecules (I⁰ on the y-axis in FIG. 11C). The relative intensities (I/I⁰) for detecting 300 μM of UA in the presence of AA, creatinine, and glucose, were 99.97%, 90.39%, and 82.59%, respectively (see FIG. 11C). Also, the relative intensities for detecting 50 μM of UA in the presence of the interfering molecules were 108.80%, 106.19%, and 103.60%, respectively (as shown in FIG. 11C). When all interferences were introduced together with UA in the electrolyte, the relative intensities compared to UA alone were 118.40% for 300 μM of UA and 120.25% for 50 μM.

Furthermore, a negative control experiment that does not include UA reveals very low ECL intensities (see FIG. 11C). In the presence of AA, creatinine, and glucose, the relative ECL intensities compared to the ECL intensity from solution with UA alone were measured to be 2.67%, 7.75%, and 5.36%, respectively. In presence of all three interferences (AA, creatinine, and glucose), the ECL intensity was 3.27% relative to the ECL intensity with UA alone. This result showed that the biosensor apparatus described herein can be specific to detecting UA. This specificity comes from the uricase, which is an enzyme that catalyzes the oxidation reaction of UA.

The biosensor apparatus can be used for the detection of UA in body fluids including saliva and urine. Normal physiological ranges of UA are 70-320 μM in saliva and 1.49-4.46 mM in urine. In order to measure the ECL intensity of UA in body fluids, these samples need to be mixed with the electrolyte. The ratio of saliva to electrolyte was chosen to be 1:1 as it is within the linear range of the biosensor apparatus proposed herein. The calculated recovery rates of UA at final concentrations were of 50 μM and 300 μM, which match the low and high ends of the normal concentrations in saliva. To account for higher concentrations of UA present normally in urine, a high concentration of UA (similar to the condition in urine) was diluted in a mixture of electrolyte and artificial urine for a final concentration of 300 μM, such that the measurement can be performed within the linear range of the device. 50 and 300 μM of UA in the electrolyte containing artificial body fluids were detected by the device (n=3) and the ECL intensities were obtained. Based on the calibration plot, the recovery rate was calculated and shown in the table below:

TABLE 1
Table showing the recovery rate for different types of bodily fluids

| | Ratio to the | UA added | UA found | RSD | Recovery |
|---|---|---|---|---|---|
| Sample | electrolyte | (□M) | (□M) | (%) | (%) |
| Saliva | 1:1 | 50 | 44.88 | 3.64 | 89.76 |
| Saliva | 1:1 | 300 | 317.77 | 2.02 | 105.92 |
| Urine | 1:1 | 50 | 41.82 | 4.09 | 83.63 |
| Urine | 1:1 | 300 | 366.29 | 4.87 | 122.10 |
| Urine | 1:9 | 300 | 334.75 | 7.75 | 111.58 |

The recovery rates for 50 μM UA in saliva and urine are 89.76% and 83.63%, with RSDs of 3.64% and 4.09%, respectively. The recovery rates for 300 μM UA in saliva and urine are 105.92% and 122.10%, with RSDs of 2.02% and 4.87%, respectively.

Since the physiological concentration of UA in human urine is higher than the linear range of UA calibration plot in this study, the effect of dilution on the recovery rate was also studied. After mixing the artificial urine with the electrolyte in 1:9 ratio, the recovery rates for 300 μM of UA is 111.58% with a RSD of 7.75%.

### COMPARISON WITH OTHER OPTICAL METHODS

To make a fair comparison between the ECL-on-CMOS platform and other platforms, including microscopes and smartphones, a Nikon eclipse Ti2 equipped with a Prime 95B 25 mm BSI CMOS camera (cooled to −25° C.), and Samsung Galaxy S21 ultra were employed. A 4× objective lens with a 30 mm working distance was chosen for the microscope to have the same field of view and capture the full length of both microfluidic channels. The imaging parameters (ISO, gain, exposure time) were chosen to be the same as the experiment with the CMOS detector. The pro imaging mode was used in the smartphone to adjust the imaging parameters to be similar to those of the other systems. The test channel in this experiment contained 300 μM H₂O₂ as the analyte. The distance between the ECL reaction and the detector was 30 mm for the microscope with a 4× objective, 300 μm for the CMOS system, and approximately 10 cm for the smartphone.

The advantage of the presently disclosed system compared to other detection systems comes from the fact that the sample is located very close to the detector, leading to a large solid angle for photon collection, as illustrated in FIG. 12C or FIG. 2. This detection solid angle is larger than the solid angle offered by most microscope objectives, which are limited by the working distance and aperture of the objective lens (FIG. 12D). In order to demonstrate that the CMOS-based ECL system has an advantage over lens-based systems, the collection efficiency of the CMOS-based SE-ECL system with other light collection systems is compared.

The biosensor apparatus was adapted to different systems, including a microscope and a smartphone, and compared the detected ECL intensities under the same experimental conditions (FIG. 12). To have a fair comparison, all the imaging parameters such as ISO (gain) and exposure time were set to be the same. Also, the magnification was chosen (4× for the microscope objective lens) such that the whole area of both channels could be visualized (FIG. 12A1, FIG. 12A2, and FIG. 12A3). ECL images of the microfluidic device are captured using different detectors (FIG. 12B1, FIG. 12B2, and FIG. 12B3), and the results of ECL intensities are compared in FIG. 12E. Overall, the ECL image captured by the CMOS shows higher intensity than either the microscope or smartphone, indicating that the CMOS platform has an overall higher efficiency for collecting photons.

In this example, it was assumed that the ECL emission is characteristic to Lambertian emitters. Therefore, the collection efficiency (η) of each platform is the fraction of the cone of light subtended by the detector divided by the whole solid angle emitted from the ECL reaction.

The angle of the detected cone of light cone and the corresponding solid angle were calculated as:

$$2 θ = Arctan ( \frac{D}{L} ) , and \\ Ω = 2 π ( 1 - cos θ ) ,$$

where 2θ is the apex angle of the cone, D is the distance between the ITO electrode (where the ECL reaction happens) and the detector (or lens), L is the diameter of the sensor (or diameter of the entrance pupil), and Ω is the solid angle subtended by the detector. Based on equation (5), when θ=π/2, the spherical cap becomes a hemisphere having a solid angle of 2π steradians. As a result, the whole ECL emission has a solid angle of 4π steradians. Therefore, the collection efficiency of the detector was calculated as:

$$η ( \% ) = ( \frac{Ω}{4 π} ) \times 100 ,$$

where η is the collection efficiency. For the microscope, D is the working distance or the focal length of the 4× lens, which is 30 mm, and θ can be calculated using the equation:

NA=n sin(θ),

where the numerical aperture (NA) of the 4× lens is 0.10, and accordingly, θ is almost 6 degrees.

Considering the whole solid angle as 360 degrees, the collection efficiency in the microscope is almost 1.7%. Using the same calculation for CMOS (this work) and measuring D=300 μm and L=3200 μm (the average length and width of the CMOS sensor), the collection efficiency is almost 40%. This calculation is not applicable for the smartphone as the focus was being changed automatically during the experiment. The main difference between these platforms is the distance between the location of the ECL reaction (from where the photons are emitted) and the detector, i.e., the distance between the ITO electrode and the detector. In the present biosensor apparatus, the reaction happens closer to the photodetector, leading to significantly higher collection efficiencies in comparison with a microscope capable of imaging a similar field of view.

Another difference between these platforms is the quantum efficiency (QE) of the imaging sensor. The sensor of the microscope (Prime 95B 25 mm BSI CMOS) has a relatively large QE (close to 95%). The CMOS sensor employed in this work (OmniVision OV5647 BSI) has a relatively lower QE of 70% (according to the manufacturer website). Considering both quantum efficiency and collection efficiency (QE and η), the microscope detector converts ˜1.6% of the ECL photons into electrons, while this number for the CMOS platform is ˜28% or almost 17.5 times higher. However, this difference is based on the calculation at room temperature. In reality, the measured improvement in the recorded ECL intensities is only a factor of approximately 10 (shown in FIG. 12E), due to additional factors such as cooling to −25° C. of the sensor used in the microscope setup that reduces the dark current.

As can be understood, the examples described above and illustrated are intended to be exemplary only. For instance, although the transparent substrate and the electrically conductive coating are described as two separate materials joined to one another, the transparent substrate and the electrically conductive coating may be made integral to one another. Moreover, it is intended that the transparency of the substrate or of the substrate is meant to encompass any type of transparency including, but not limited to, semi, partial or full transparency at some or all of the relevant wavelengths. The scope is indicated by the appended claims.

What is claimed is:

1. A biosensor apparatus comprising: a photodiode array having a first photon receiving face, a transparent substrate covering the first photon receiving face of the photodiode array, the transparent substrate having a second photon receiving face opposite the photodiode array, an electrically conductive coating covering the second photon receiving face of the transparent substrate, the electrically conductive coating being transparent and having two electrical contacts spaced apart from one another, and an analyte receiving area extending between the two electrical contacts of the electrically conductive coating.

2. The biosensor apparatus of claim 1 further comprising a body of material deposited onto the transparent substrate, the body having a recessed channel forming a fluidic channel when the recessed channel is received onto the analyte receiving area.

3. The biosensor apparatus of claim 2 wherein the body has an inlet and an outlet fluidly connected at opposite ends of the fluidic channel.

4. The biosensor apparatus of claim 1 wherein the photodiode array, the transparent substrate and the electrically conductive coating form a first detector assembly, the biosensor apparatus comprising a second detector assembly identical to the first detector assembly, the first detector assembly and the second detector assembly facing the analyte receiving area to receive photons therefrom.

5. The biosensor apparatus of claim 4 wherein the first detector assembly and the second detector assembly form a gap therebetween, the gap having a dimension ranging between 50 μm and 200 μm.

6. The biosensor apparatus of claim 1 wherein the photodiode array is a complementary metal-oxide-semiconductor (CMOS) sensor.

7. The biosensor apparatus of claim 1 wherein the electrically conductive coating has a thickness between 3 nm and 500 nm, and most preferably between 10 nm and 300 nm.

8. The biosensor apparatus of claim 1 wherein the electrically conductive coating is an Indium Tin Oxide (ITO) coating.

9. The biosensor apparatus of claim 1 wherein the transparent substrate has a thickness between 50 nm and 200 μm, and most preferably between 100 μm and 150 μm.

10. The biosensor apparatus of claim 1 wherein the transparent substrate is a Poly Ethylene Terephthalate (PET) film.

11. The biosensor apparatus of claim 10 wherein the PET film forms a 3-dimensional pattern having a plurality of protrusions protruding from the photodiode array.

12. The biosensor apparatus of claim 11 further comprising an external voltage source applying an electrical voltage across the electrically conductive coating via the two electrical contacts.

13. The biosensor apparatus of claim 12 further comprising a pair of conductive connectors electrically connected between the two electrical contacts and the external voltage source.

14. A method of optically detecting an analyte using a biosensor apparatus, the biosensor apparatus having a photodiode array, a transparent substrate atop the photodiode array, an electrically conductive coating atop the substrate, the electrically conductive coating being transparent and having two electrical contacts spaced apart from one another, and an analyte receiving area atop the electrically conductive coating between the two electrical contacts, the method comprising:
the analyte receiving area receiving an analyte containing sample;
generating an electromagnetic field across the analyte receiving area via the two electrical contacts, said electromagnetic field stimulating electrochemiluminescence (ECL) light emission in the analyte containing sample; and
the photodiode array generating an electrical signal based on a detection of said ECL light emission.

15. The method of claim 14 wherein the analyte containing sample is one of liquid and gaseous.

16. The method of claim 14 wherein said generating the electromagnetic field includes applying an electrical voltage along the electrically conductive coating between the two electrical points.

17. The method of claim 16 wherein the electrical voltage is of at least 5 V.

18. The method of claim 14 wherein said receiving the analyte containing sample includes flowing the analyte containing sample within a fluidic channel extending across the analyte receiving area.

19. A biosensor apparatus comprising: a photodiode array, a substrate atop the photodiode array, an electrically conductive coating atop the substrate, and an analyte receiving area atop the electrically conductive coating, the photodiode array having a field of view encompassing the analyte receiving area and extending across the substrate and the electrically conductive coating.

20. The biosensor apparatus of claim 19 wherein when an electrical voltage is applied across the electrically conductive coating, analytes present at the analyte receiving area emit electrochemiluminescence (ECL) light emission across the electrically conductive coating, across the substrate and towards the photodiode array.
